(19) United States
(12) Patent Application Publication
(10) Pub. No.: US 2026/0108346 A1
(43) Pub. Date: Apr. 23, 2026

(54) FIXATION SYSTEM AND FIXATION KIT FOR LIGAMENTOPLASTY

(71) Applicants: NOVETECH SURGERY S.A.R.L., MONACO (MC); UNIVERSITE CLAUDE BERNARD LYON 1, Villeurbanne Cedex (FR); INST ENS SUP RECH ALIMENTATION SANTE ANIMALE SCIENCES AGRONOMIQUES ET ENVIRONNEMENT (VETAGRO SUP), Marcy L’Etoile (FR); UNIVERSITE GUSTAVE EIFFEL, Marne-la-Vallee Cedex (FR)

(72) Inventors: Bastien GOIN, Le Cannet (FR); Eric VIGUIER, Tassin la Demi-Lune (FR); Thibaut CACHON, Sainte Consorce (FR); Michel MASSENZIO, Lentilly (FR); Yoann LAFON, Villeurbanne (FR); Victor ROBALO, Nice (FR); Damien LEFIEF, Poulx (FR); Philippe BUTTIN, Villaz (FR)

(21) Appl. No.: 19/487,001

(22) PCT Filed: May 23, 2024
(86) PCT No.: PCT/EP2024/064205

§ 371 Date: Nov. 24, 2025

(30) Foreign Application Priority Data
May 24, 2023 (FR) FR2305140

(51) Int. Cl. A61F 2/08

(52) U.S. Cl. CPC A61F 2/0811; A61F 2002/0829; A61F 2002/0864; A61F 2002/0882; A61F 2250/0012

(57) ABSTRACT

The invention pertains to a fixation system for ligamentoplasty including a cage including a longitudinal borehole including a proximal end and a distal end, an outer surface including a thread, a distal portion wherein the borehole is tapered, a medial portion, an internal part designed to be inserted into the opening of the cage including a tapered distal part, a central part, a proximal part including a thread, wherein the cage includes a proximal portion with a cross-section larger than that of the medial portion thereby forming a collar, and wherein the borehole has a cross-section larger than that of the medial portion, the borehole including a thread over an angular sector of less than 360° (a portion of its circumference) designed to engage with the internal part.

### TECHNICAL FIELD OF THE INVENTION

The present invention relates to the field of ligament reconstruction. The invention will be particularly applicable for the reconstruction of the cruciate ligament, preferably the cranial cruciate ligament (CCL) in animals, but it may also be used in humans, particularly for the cruciate ligaments of the knee.

### PRIOR ART

In the event of rupture of the cranial cruciate ligament, ligamentoplasty is currently rarely performed in veterinary medicine. This is due to the etiopathogenesis of the injury and the varying morphological specificities in the animal. Additionally, there are challenges related to tools and implants that are not suitable.

In human medicine, it is well established that damaged anterior cruciate ligaments can be repaired by grafting a tendon graft that is physiologically attached to bone blocks at each end, situated between the femur and tibia in the knee joint. The graft is placed into a prepared hole and initially secured with a screw inserted into the hole. After a while, the graft either heals or integrates successfully.

Although this technique generally works well, issues can arise when the screw head rests against the graft material; when the knee is bent and the graft rotates, the screw head can serve as a support point for the graft, potentially damaging the grafted fibers. Other problems may arise as the threads of the screws used cut and thus damage the graft. Other technical limitations of this system include the compression caused by the bone/tendon/screw fixation system, which contributes to the ovalization of the bone tunnel due to necrosis of bone cells from excessive mechanical stress and the infiltration of synovial fluid into the bone tunnel. Although the impact on the fixation is minor, it presents challenges for surgical revision due to diminished bone mass.

Additionally, screws may be lost in the knee joint cavity during certain procedures, resulting in a longer operation than necessary and causing further trauma.

A recognized surgical technique used in ACL reconstruction in a damaged knee joint involves the use of a dedicated interference screw; however, it is highly dependent on bone quality, which poses a barrier to the advancement of this technique.

The aim of this technique is to reconstruct the ligament using the tendons from the patient's hamstring muscles, which are inserted into the knee joint through small bone tunnels created in the tibia and femur.

Suspension tapes positioned at each end of the ligament graft are commonly used for fixation in pre-prepared bone tunnels.

Fixing the tapes rather than the graft in the bone tunnels provides high tensile strength and minimizes the risk of graft pull-out or slippage, which at least partially limits the phenomenon of bone necrosis by eliminating bone/tendon/screw interference fixation.

The technique for preparing a graft is well known to the person skilled in the art.

It is known to use interference screws that are inserted into the bone tunnels to hold the suspension tapes in position.

Many interference screws are already known. However, these screws used to secure the tapes or grafts against the wall of the bone tunnel have the drawback of increasing the risk of damaging the bone of the tunnel, which must endure the repeated stresses from the tapes or grafts over time, and they do not address the issue posed by the interference fixation system by changing a bone/tendon/screw fixation into a bone/tape/screw sandwich fixation.

Devices for ligamentoplasty are also known which, in addition to an interference screw, comprise a cage designed to be anchored in the bone tunnel, and into which the tapes or grafts are inserted, along with the interference screw, which helps to minimize bone weaknesses by controlling the interface between the components.

Document FR2924596 describes a fixation device for ligamentoplasty comprising a hollow cage with an internal borehole opening at both ends of said cage and an outer wall featuring a threaded section, along with a locking member designed to fit into the internal borehole of the cage, characterized by the fact that the thread consists of soft threads, and the outer wall of the cage terminates at the upper end either with the thread or with a smooth section that has an outer diameter less than or equal to the inner diameter of said thread. The cage has a cylindrical outer shape allowing it to be fully inserted into the formed bone tunnel. The inner wall of the borehole of the cage comprises a thread in the proximal part designed to engage with the thread of the locking member. The tapes or grafts pass through the cage within the borehole. The inner wall comprises two longitudinal grooves serving as housing for the tapes. Although this device has improved outcomes in ligamentoplasty, it still has weaknesses, including the risk of rupture or slippage of the suspension tapes over time, particularly at the tightening points between the cage and the locking member. Specifically, the fixation point is proximal, which limits the rigidity of the reconstruction and the two technical functions provided by the threading of the counter screw—namely, securing the tape with the counter screw and locking the counter screw within the cage under cyclic loads—entail a risk of loosening of the counter screw. The aim of the present invention is to propose a solution that makes it possible to facilitate the installation of the system and to optimize its durability over time.

The other objects, features and advantages of the present invention will be clear after an examination of the following description and the accompanying drawings. It is understood that other advantages can be incorporated.

### SUMMARY OF THE INVENTION

To achieve this objective, according to one embodiment a ligamentoplasty fixation system is provided, comprising:
A cage comprising
a longitudinal borehole comprising a proximal opening end and a distal opening end,
an outer surface comprising a thread,
a distal portion in which the borehole is tapered,
a medial portion,
an internal part designed to be inserted into the holes of the cage, comprising
a tapered distal portion,
a central part,
a proximal portion comprising a thread,
   characterized in that the cage comprises:
a proximal portion with a cross-section larger than that of the medial portion, thereby forming a collar, and wherein the borehole has a cross-section larger than that of the medial portion, the borehole comprising a thread over an angular sector of less than 360° designed to engage with the internal part.

This system thus allows for better initial and long-term stability, thanks to effective anchoring of the cage, particularly due to the collar that ensures close contact with the bone surface, as well as the presence of a partial engagement thread between the cage and the internal part in the proximal portion, which reduces stress on the graft. Furthermore, this thread configuration in the proximal portion creates additional space in this area, making it easier to insert the internal part into the holes of the cage, even in the presence of the graft, without any effort or risk of damaging it.

Advantageously, the proximal portion of the cage comprises an enlargement of the hole at the proximal end within the additional angular sector comprising the thread, thereby creating a notch. This arrangement further increases the volume available for the graft in this area to facilitate insertion of the internal part and tightening without damaging the graft.

The system according to the invention ensures distal fixation which avoids the drawbacks of the prior art. The system increases the stiffness of the reconstruction by the nearest fixation point in the joint.

In addition, the system ensures fixation without impacting the graft.

Another aspect relates to a drive system of the cage comprising an additional distal end of the proximal portion of the cage having a section with a cylindrical area which corresponds to the angular sector provided with the thread and an oblong area corresponding to the angular sector having no thread.

Another aspect of the invention pertains to an ancillary device comprising at least one connecting element to the proximal portion of the cage and a distal opening designed to guide a tool for securing the internal part.

Another aspect of the invention pertains to a kit for ligamentoplasty comprising a system as described above and/or a cage drive system and/or an ancillary device and/or a tool for guiding the internal part.

### BRIEF DESCRIPTION OF THE FIGURES

The purposes, objects, characteristics and advantages of the invention will be more apparent from the detailed description of one embodiment thereof which is illustrated by the following accompanying drawings wherein:

FIG. 1 shows a longitudinal sectional view of the system according to the invention.

FIG. 2 shows a longitudinal sectional view of the system according to FIG. 1 comprising the graft positioned between the cage and the internal part.

FIG. 3 shows a side view of the cage of the system according to the invention.

FIG. 4 shows a longitudinal sectional view of the cage of the system according to the invention.

FIG. 5 shows a proximal view of the cage of the system according to the invention.

FIG. 6 shows a distal view of the cage of the system according to the invention.

FIG. 7 shows a side view of the internal part of the system according to the invention.

FIG. 8 shows a longitudinal sectional view of the internal part of the system according to the invention.

FIG. 9 shows a proximal view of the internal part of the system according to the invention.

FIG. 10 shows a perspective view of the drive system of the cage of the system according to the invention.

FIG. 11 shows a longitudinal sectional view of the drive system of the cage of the system according to the invention.

FIG. 12 shows a side view of the drive system of the cage of the system according to the invention.

FIG. 13 shows a distal view of the drive system of the cage of the system according to the invention.

FIG. 14 shows a perspective view of the right side of the ancillary device according to the invention.

FIG. 15 shows a perspective view of the left side of the ancillary device according to the invention.

FIG. 16 shows a view of the right side of the ancillary device according to the invention.

FIG. 17 shows a view of the left side of the ancillary device according to the invention.

FIG. 18 shows a bottom view of the ancillary device according to the invention.

FIG. 19 shows a top view of the ancillary device according to the invention.

FIG. 20 shows a perspective view of the right side of the ancillary device to which the cage is attached.

FIG. 21 shows a perspective view of the left side of the ancillary device to which the cage is attached.

FIG. 22 shows a view of the left side of the ancillary device to which the cage is attached.

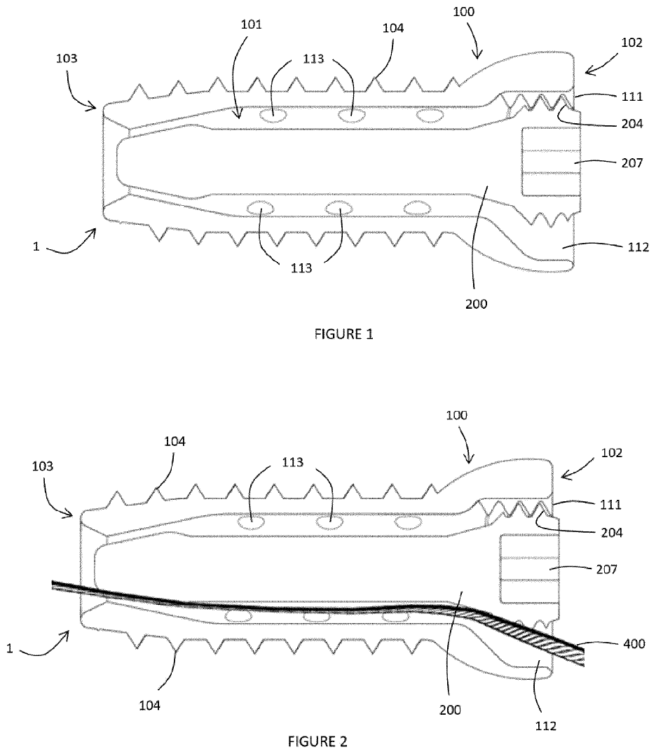

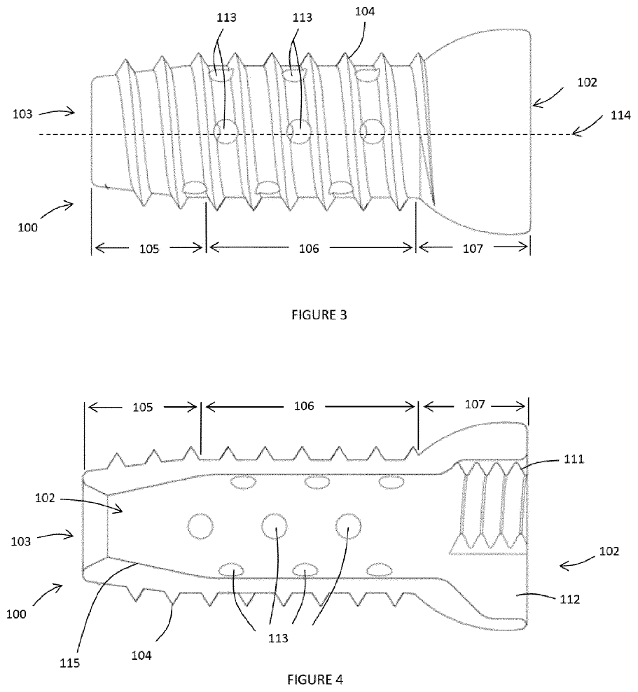

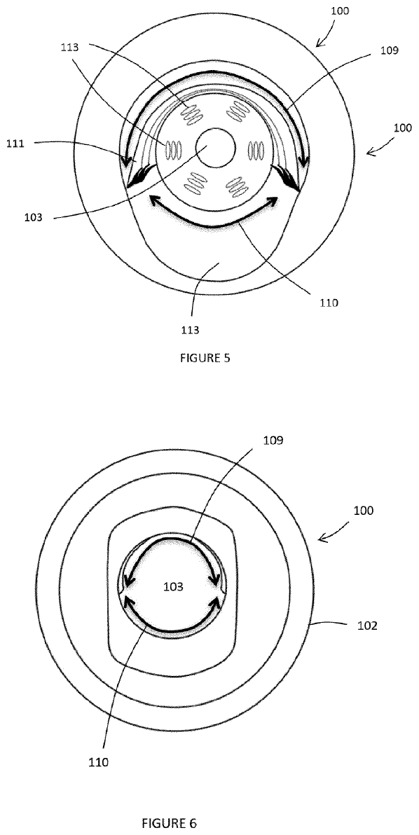

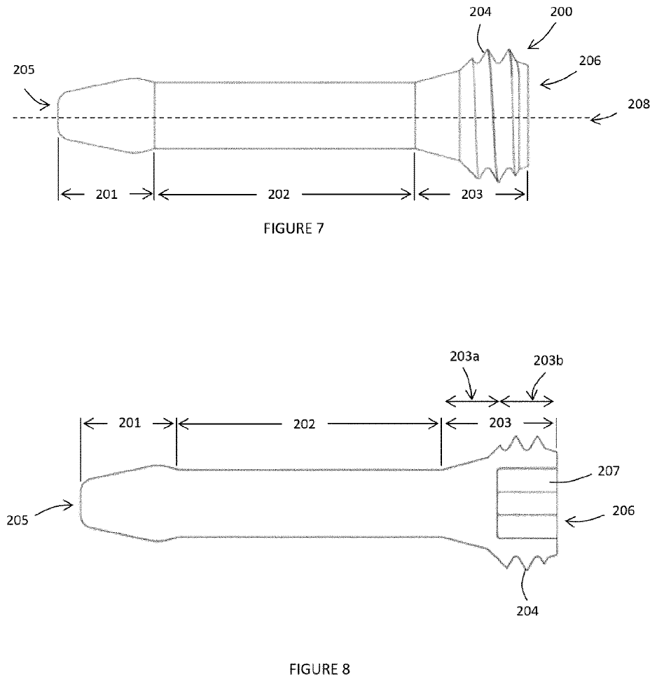

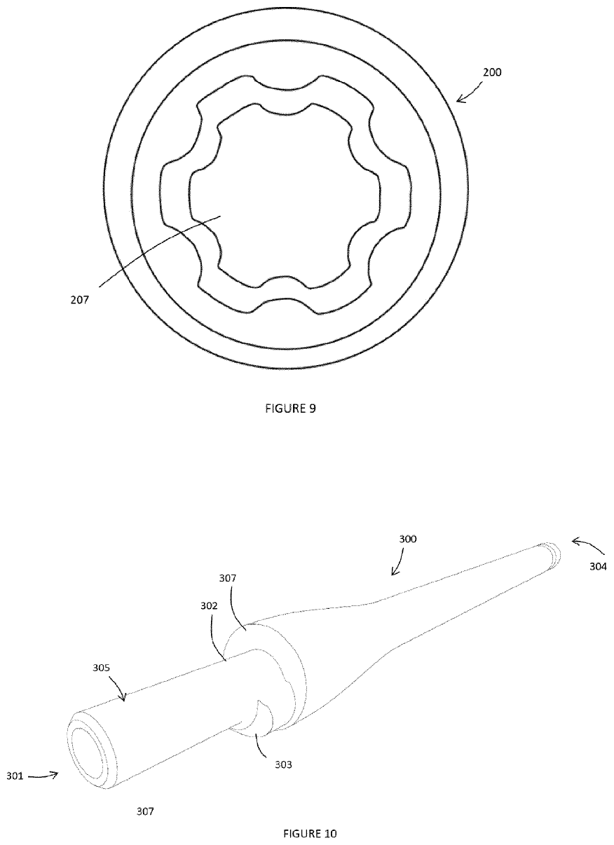

The drawings are given as examples and do not limit the invention. They constitute schematic representations intended to facilitate understanding of the invention and are not necessarily plotted to the scale of practical applications.

### DETAILED DESCRIPTION OF THE INVENTION

Before undertaking a detailed review of embodiments of the invention, optional characteristics are set out below that can optionally be used in combination or alternatively:
According to one example, the proximal portion 107 of the cage 100 has an outer surface that has no thread;
According to one example, the proximal portion 107 of the cage 100 comprises an enlargement of the borehole 101 at the proximal end 102 within an additional angular sector 110 of the angular sector 109 that contains the thread 111, thereby forming a notch 112;
According to one example, the angular sector 109 of the borehole 101 comprising the thread 111 ranges from 90° to 270°, preferably from 100° to 250°, and preferably around 240°;
According to one example, the borehole 101 is bored in the distal portion 105 and in the medial portion 106;
According to one example, the distal part 201 of the internal part 200 is smooth;
According to one example, the central part 202 of the internal part 200 is at least partially cylindrical and smooth, and it has a smaller cross-section than that of the distal part 201;
According to one example, the proximal part 203 of the internal part 200 has a larger cross-section than that of the central part 202; the notch is unobstructed by the internal part when it is inserted into the cage;
According to one example, the cage 100 comprises fenestrations 113 designed for the osseointegration of the system; preferably the fenestrations are approximately one millimeter in size;
According to one example, the ancillary device 500 comprises a plurality of connecting elements arranged around the perimeter of the distal opening, and the cage 100 comprises a plurality of additional connecting elements designed to engage with the plurality of connecting elements;
According to one example, the ancillary device 500 comprises a device for tensioning a graft 400.

The terms “proximal” and “distal” are understood in relation to the surgeon handling the fixation system. “Proximal” refers to what is closer to the surgeon, while “distal” refers to what is further away from the surgeon. Both the right and left are also understood in relation to the surgeon when the system, the ancillary device, and the drive system are in use. The right side of the ancillary device refers to the portion that is located to the right of the surgeon when they are handling the ancillary device.

Vertical refers to what is parallel to the direction of gravity, as indicated by a plumb line, while horizontal refers to what is perpendicular to the vertical. The top and bottom are vertically opposite.

Horizontal means perpendicular to the vertical.

“Transverse” means a direction perpendicular to a longitudinal axis. A cross section is a section perpendicular to the longitudinal axis.

A parameter described as “substantially equal to/greater than/less than” or “about” a given value means that this parameter is equal to/greater than/less than the specified value, within a margin of plus or minus 10%, or even plus or minus 5%, of that value.

For purposes of the present disclosure, the term “A and/or B” means (A), (B) or (A and B). For the purposes of the present disclosure, the term “A, B and/or C” means (A), (B), (C), (A and B), (A and C), (B and C), or (A, B and C).

The present invention relates to a fixation system for ligamentoplasty fixation system. The system according to the invention is intended in particular for a veterinary application. The system is designed to facilitate the fixation of a biological implant, also known as a natural implant, or a synthetic one, for the surgical treatment of chronic or traumatic tendon or ligament conditions. In this application, the term graft 400 refers to synthetic ligament grafts, biological ligament grafts, synthetic tendon grafts, and biological tendon grafts. Preferably, the system is intended for attaching a single graft 400. The graft 400 advantageously presents as a tape. The tape can take the form of a braid that may be either round or flat.

The system according to the invention comprises two cooperating parts: a cage 100 and an internal part 200. Advantageously, the two parts function as a counter screw, enabling the graft 400 to be secured with a second, smaller gauge screw.

The cage 100 comprises a distal end 103 and a proximal end 102. The longitudinal axis 114 of the cage 100 passes through the two distal 103 and proximal 102 ends of the cage.

The cage 100 comprises a borehole 101 that advantageously extends through the cage in its largest dimension and preferably along its longitudinal axis 114. The borehole 101 is advantageously centered in the cage 100. According to one possibility not shown, the borehole 101 is off-center in the cage 100. The borehole 101 preferably opens at each end of the cage: at the proximal end 102 and at the distal end 103. The borehole is a tunnel running right through the cage 100. The cage 100 is hollow. The cage is a female part.

Advantageously, the cage 100 is configured to allow the introduction of the internal part 200 into said cage 100 and more precisely into the borehole 101. The borehole 101, for instance, has a circular cross-section, except for the proximal portion 107. The borehole 101 creates an inner surface 115 of the cage 100.

The cage 100 comprises an outer surface advantageously designed to come into contact with the body of an animal or human, more precisely with the bone into which the system is intended to be inserted. The cage 100 is advantageously inserted using a drive system 300 into a pre-drilled hole in the bone. The outer surface of the cage advantageously features a rough texture that optimizes bone anchorage, for example with a roughness Ra ranging from 1.6 to 20.

The cage 100 features a cross-section designed to facilitate the screwing of the cage 100. Preferably, the cage 100 features a circular outer cross-section.

According to one embodiment, the outer surface comprises a thread 104. Advantageously, the outer surface is only partially covered with the thread 104. The thread 104 is intended to provide anchorage in the bone. The thread 104 is preferably aggressive or sharp to ensure secure anchoring, particularly in cancellous bone.

According to one embodiment, the cage 100 comprises a distal portion 105 that extends from the distal end 103 toward the proximal end 102 and advantageously up to a medial portion 106. The distal portion 105 of the cage 100 comprises the borehole 101, which has a conical shape in this distal portion 105. The borehole 101 features a cross-section that narrows toward the distal end 105. According to one embodiment, in the distal portion 105, the inner surface 115 of the cage 100 formed by the borehole 101 has an inclination between 5° and 80°, preferably between 12 and 20°, relative to the longitudinal axis. This inclination is preferably directed toward the outside of the cage and toward the proximal end 102. This inclination is strategically selected to ensure continuous contact for optimal fixation of the graft 400 while minimizing friction with the graft 400.

According to one possibility, the thread 104 of the outer surface of the cage 100 extends at least partially over this distal portion 105. According to one possibility, the distal portion 105 has, a circular outer cross-section.

According to one embodiment, the cage 100 comprises a medial portion 106 that extends from the distal portion 105 toward the proximal end 102 and advantageously up to a proximal portion 107. The thread 104 extends advantageously over the outer surface of this medial portion 106 of the cage 100. The medial portion 106 of the cage comprises the borehole 101, which features an advantageously constant internal cross-section in this medial portion 106. According to a preferred embodiment, the medial portion 106 comprises an inner cross-section with a diameter larger than that of the internal part 200 designed to be inserted into the cage 100. This allows the internal part 200 to slide into the borehole 101 while also creating a space for the graft 400 to be positioned. According to another possibility not shown, the medial portion 106 comprises a groove that extends longitudinally within the borehole. Preferably, the system comprises a single groove, which is designed to accommodate the graft 400. Advantageously, the inner cross-section of the medial portion 106 is larger than or equal to the largest inner cross-section of the distal portion 105.

According to a preferred embodiment, the inner surface 115 of the cage extends into both the distal portion 105 and the medial portion 106, ensuring that the change in inclination between the distal portion 105 and the medial portion 106 is smooth and free of sharp angles. The acute angle formed between the inner surface 115 at the distal portion 105 and the inner surface at the medial portion 106 is greater than 90°, preferably in the range of 140 to 170°.

According to one embodiment, the cage 100 comprises a proximal portion 107 that extends from the proximal end 102 toward the distal end 103, preferably up to the medial portion 106. Preferably, the proximal portion 107 does not comprise a thread 104. Preferably, the thread 104 stops at the junction between the medial portion 106 and the proximal portion 107.

The proximal portion 107 advantageously has a collar. The collar is formed by a cross-section of the outer surface of the cage 100 greater than that of the medial portion 106. According to one possibility, the cross-section of the outer surface of the cage 100 increases in the direction of the proximal end 102. The collar shape of the proximal portion 107 makes it possible to provide cortical support to the system. The proximal portion 107 preferably widens toward the proximal end 102, and this widening creates the collar. According to a preferred embodiment, the collar is designed not to be fully embedded in the bone. Preferably, at least the proximal end of the proximal portion 107 remains above the surface of the bone.

The proximal portion 107 of the cage comprises the borehole 101, which has an inner cross-section in this portion that is larger than that of the medial portion 106. The borehole 101 comprises in this proximal portion 107 a thread 111 extending over an angular sector 109 of the borehole 101 less than 360°. Thus, the borehole 101 comprises a portion of its circumference that does not have a thread 111; it is an additional angular sector 110 without a thread 111. The absence of thread 111 on an angular sector 110 helps prevent damage to the graft 400, which is advantageously positioned within this angular sector 110, while the angular sector 109 is designed to engage with an additional thread 204 of the internal part 200. Advantageously, the thread 111 extends over a single angular sector of the borehole 101 less than 360°. Only a portion of the circumference of the borehole 101 does not have a thread 111. The borehole 101 comprises a single angular sector less than 360° comprising the thread 111 and a single portion not comprising the thread 111. The portion not comprising a thread 111 and the angular sector comprising the thread represent an angular sector of 360°.

According to a preferred embodiment, the proximal portion 107 comprises an enlargement of the borehole 101 in the additional angular sector 110 that has no thread 111. This enlargement forms a notch 112 helping to allow passage of the graft 400 without damaging it.

Advantageously, the angular sector 109 comprising the thread 111 extends over an angular sector less than or equal to 300°, preferably less than or equal to 270°, preferably less than or equal to 250°. Advantageously, the angular sector 109 comprising the thread 111 extends over an angular sector greater than or equal to 90°, preferably greater than or equal to 100°, preferably greater than or equal to 180°. Advantageously, the angular sector 109 comprising the thread 111 extends over an angular sector of 90° to 300°, preferably of 100° to 270°, preferably of 180° to 250°. Preferably, the angular sector 109 is continuous or uninterrupted, i.e. it is formed of a single part and not of a plurality of sections the sum of which would correspond to a total angular sector less than or equal to 300°.

The thread 111 is designed to engage with the internal part 200.

According to a preferred embodiment, the cage 100 comprises fenestrations or lights 113 that pass through the wall of the cage 100, thereby connecting the borehole 101 to the outer surface of the cage 100. The lights 113 have a diameter of about one millimeter. The lights 113 enable better cell circulation, promoting effective osseointegration of the cage 100.

The borehole 101 is advantageously bored, particularly in the distal portion 105 and preferably also in the medial portion. The borehole 101 is therefore smooth at least in this or these portions. This borehole prevents any damage to the graft 400.

At the distal end 103 of the cage 100, the borehole 101 has an oblong opening, meaning its cross-section is preferably elongated, with edges that are rounded or softened to facilitate the passage of the implant without causing damage.

The invention comprises an internal part 200. The internal part 200 is designed to be inserted into the cage 100, and more precisely into the borehole 101. The primary function of the internal part 200 is to secure the graft 400 within the cage 100. Advantageously, the internal part 200 is a solid part.

The internal part 200 has a distal end 205 and a proximal end 206. The longitudinal axis 208 of the internal part 200 runs through both the distal end 205 and the proximal end 206 of the internal part. According to one embodiment, the longitudinal axis 114 of the cage 100 and the longitudinal axis 208 of the internal part 200 are parallel or even coincide. According to another embodiment, the longitudinal axis 208 of the internal part 200 is misaligned with the longitudinal axis 114 of the cage when the graft 400 is secured between the cage 100 and the internal part 200. Indeed, this securing mechanism compresses the graft 400 between the inner surface 115 of the cage and the internal part 200, causing a slight misalignment of the internal part 200, particularly its distal part 201, which thus also comes into contact with or compresses against the inner surface 115 of the cage opposite the graft 400.

According to another embodiment, the longitudinal axis 114 of the cage and the longitudinal axis 208 of the internal part 200 are parallel, but do not coincide. This arrangement is possible when the borehole 101 is not centered in the cage 100, particularly when the cage has a single groove formed parallel to the borehole 101.

The internal part 200 comprises a distal part 201 that extends from the distal end 205 toward the proximal end 206, and advantageously up to a central part 202. The distal portion 205 is advantageously tapered in shape. The distal part 201 features a cross-section that widens from the distal end 205 toward the proximal end 206. The distal part 201 is designed to fit into the distal portion 105 of the cage 100.

The internal part 200 comprises a central part 202 that extends from the distal part 201 toward the proximal end 206 and advantageously toward the proximal part 203. The central part 202 has a cross-section that is preferably constant. The cross-section of the central part 202 is less than or equal to the largest cross-section of the distal part 201. If the cross-section of the central part 202 is smaller than the largest cross-section of the distal part 201, this creates a space between the cage 100 and the internal part 200, specifically between the medial portion 106 and the central part 202. This configuration allows more space for the graft in this medial portion 206.

The internal part 200 comprises a proximal portion 203. The proximal portion 103 extends from the proximal end 205 toward the distal end 205 and advantageously toward the central part 202. The proximal part 203 conveniently comprises a thread 204. The thread 204 is advantageously formed on at least part of the proximal portion 203. The thread 204 is designed to engage with the thread 111 of the cage 100. Preferably, the proximal portion 203 has a first subpart 203a with a tapered shape that preferably has no thread. The first subpart 203a extends from the central part 202 to the second subpart 203b. The second subpart 203b extends from the first subpart 203a toward the proximal end 206 and advantageously has a constant circular cross-section.

The distal portion 203 comprises an indent 207 advantageously formed at the proximal end 206. The indent 207 is designed and configured to work with a tool for installing the internal part 200 in the cage 100.

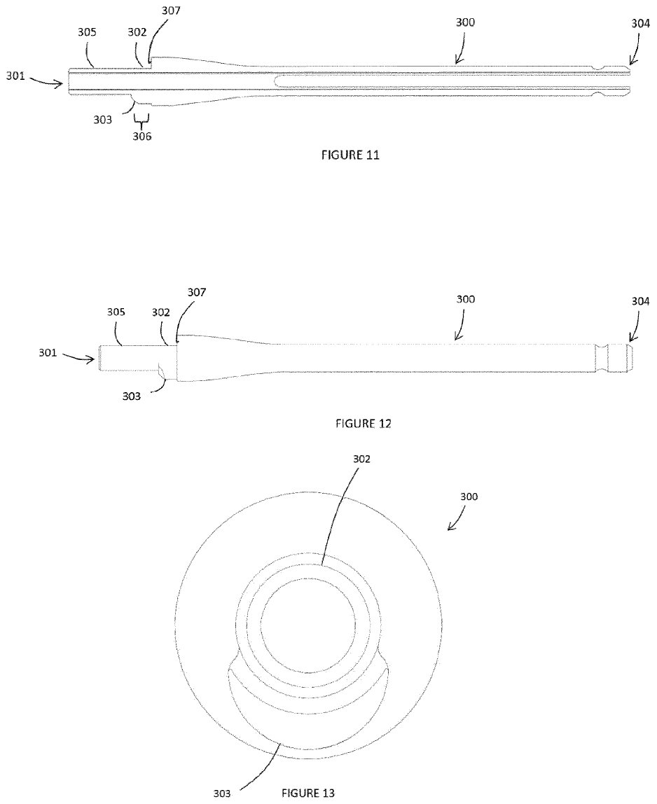

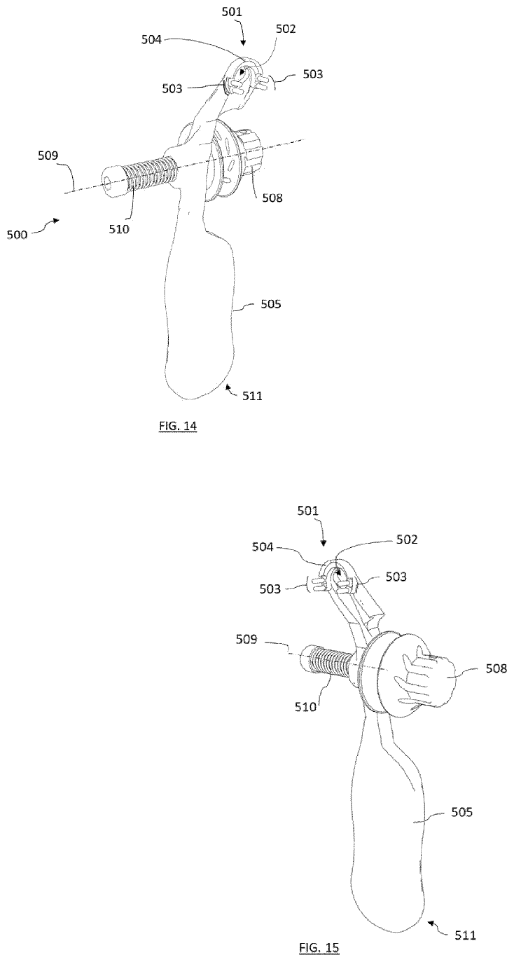

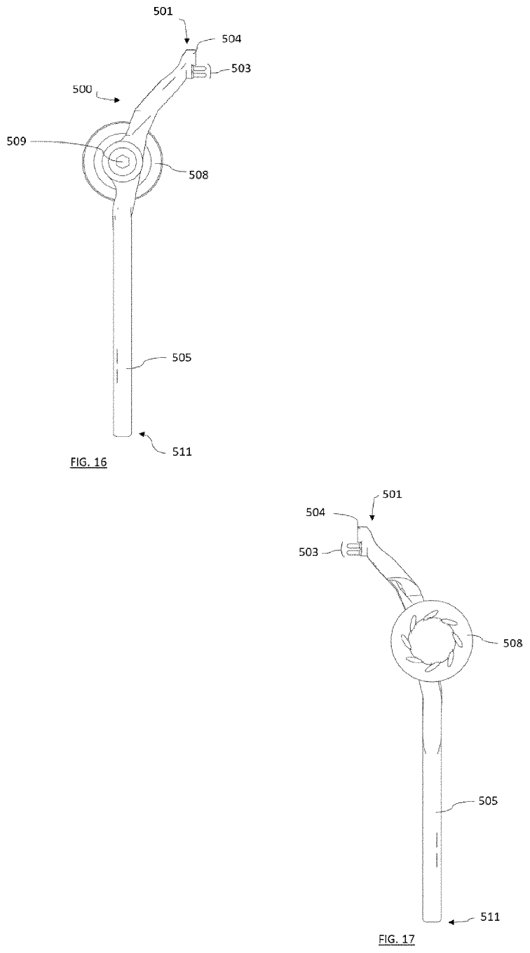

According to one aspect, the invention relates to a method for fixing a system according to the invention for ligamentoplasty. The method advantageously comprises drilling a hole in the bone. The process involves inserting the cage 100 into a hole that has been previously drilled in the bone. The setup is advantageously carried out by the drive system 300 as described above and illustrated in FIGS. 10 to 13. Once the cage 100 has been implanted, the graft 400 is passed through the cage 100, specifically through the borehole 101. The graft 400 is advantageously positioned at the proximal end 102 within the notch 112 of the proximal portion 107. The internal part 200 is then positioned in the cage 100 preferably using a tool, not shown, designed to fit into the indent 207 of the internal part 200. The distal end 205 of the internal part 200 is inserted into the borehole 101, preferably while keeping the graft in the notch 112. The proximal part 203, and more specifically the thread 204, advantageously of the subpart 203b, engages with the thread 104 of the proximal portion 107. The internal part 200 is screwed into the cage 100. The internal part 200, and more specifically the distal part 201, is applied to the graft 400 which is itself applied to the inner surface 115 of the distal portion 105 of the cage 100, thus ensuring the fixation of the graft 400.

The system according to the invention thus secures the graft 400 within the system by means of compression between the cage 100 and the internal part 200.

According to one embodiment, the cage 100 and the internal part 200 are made of metal, such as titanium, stainless steel, or polyether ether ketone (PEEK). The cage 100 and the internal part 200 are for example manufactured by machining, molding, or additive manufacturing such as 3D printing. This enables the production of internal structures with complex curves, as well as the creation of a rough surface ideal for bone anchorage or for cancellous and/or porous structures.

According to one aspect, the invention pertains to a drive system 300 designed to engage with the cage 100 to ensure its placement in a hole in a bone.

According to one aspect, the invention relates to a kit comprising the fixation system for ligamentoplasty comprising the cage 100 and the internal part 200 as well as the drive system 300.

Advantageously, the drive system 300 for installing the cage 100 is cannulated so as to facilitate the implantation of the system along the axis of the pre-drilled bone.

The drive system 300 designed for the installation of the cage 100 is illustrated in FIGS. 10 to 13.

The drive system 300 comprises a distal end 301 designed to engage with the proximal end 102 of the cage 100 and a proximal end 304 that is advantageously designed to engage with a gear motor for the rotational actuation of the tool 300.

The drive system 300 comprises an additional area 305 of the borehole section 101, and more specifically the medial portion 106. The additional area 305 advantageously features a circular cross-section of a constant diameter. This additional area 305 has a maximum length corresponding to the length of the medial portion 106. This area 305 extends from the distal end 301 preferably to the intermediate area 306. The intermediate area 306 is designed to engage with the proximal portion 107 of the cage 100. The intermediate area 306 comprises a first area called oblong area 303 and a second area called circular area 302. The oblong area 303 extends over an angular sector corresponding to the angular sector 110 additional to the angular sector 109 of the cage 100. The circular area 302 extends over an angular sector corresponding to the angular sector 109 of the cage 100. The circular area 302 features a circular outer surface that extends from the additional area 305. The oblong area 303 has a prominent circular outer surface relative to the additional area 305. Preferably, the tool 300 comprises a stop surface 307 designed to be applied to the proximal end 102 of the cage 100.

According to one possibility, the invention comprises a tool for installing the internal part 200. This tool is not shown in the figures. The tool for installing the internal part 200 is designed to engage with the indent 207 of the internal part 200. The tool for installing the internal part 200 may be included in a ligamentoplasty kit.

According to one aspect, the invention pertains to an ancillary device 500 designed to engage with the cage 100 for installing the internal part 200 and tensioning the graft 400.

According to one aspect, the invention pertains to a kit comprising the fixation system for ligamentoplasty, comprising the cage 100 and the internal part 200, as well as the ancillary device 500, and advantageously the drive system 300, and possibly the tool for installing the internal part 200.

The ancillary device 500 comprises a distal part 501 and a proximal part 511 forming a handle 505 intended to be grasped by the surgeon. For example, the handle 505 is designed with a flattened shape to facilitate its handling.

The distal part 501 and the proximal part 511 join at a clamping assembly. The clamping assembly comprises a transverse opening formed in the ancillary device that accommodates a clamping axis 509; at one end of the clamping axis 509, there is a hand-operated clamping knob 508, and at the opposite end there is a retaining counter spring 510. Advantageously, the clamping axis 509 comprises a slot 507 configured and designed to receive the graft 400, ensuring its tensioning by operating the clamping knob 508.

The distal part 501 comprises an opening 502. The opening 502 is advantageously perpendicular to the opening receiving the clamping axis 509. The opening 502 is designed to accommodate at least the tool for installing the internal part 200. According to one option, the opening 502 is also designed to allow the passage of the internal part 200.

The distal part 501 is configured to engage with the cage 100, and more specifically with the proximal end 102. The distal part 501 is designed to work with the cage 100 to keep it securely in place within the bone tunnel during the insertion and tightening of the internal part 200. The distal part 501 comprises at least one connecting element designed to engage with the cage 100. Preferably, the cage 100 comprises an additional connecting element designed to engage with the connecting element of the ancillary device 500. The connecting element is configured to prevent rotation of the cage 100 during rotation of the internal part 200.

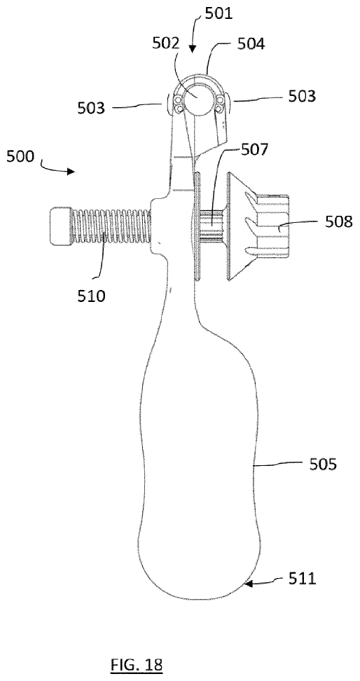

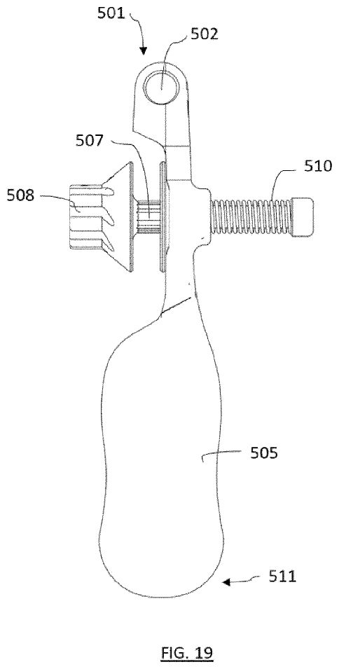

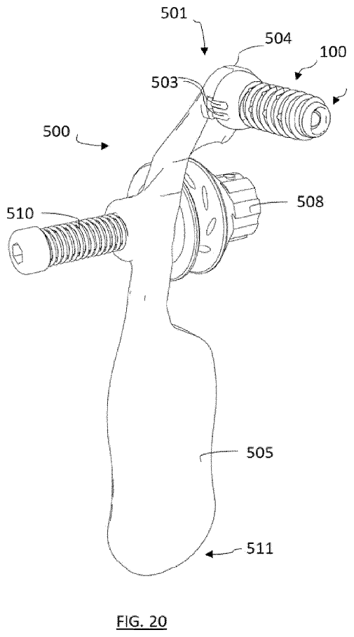

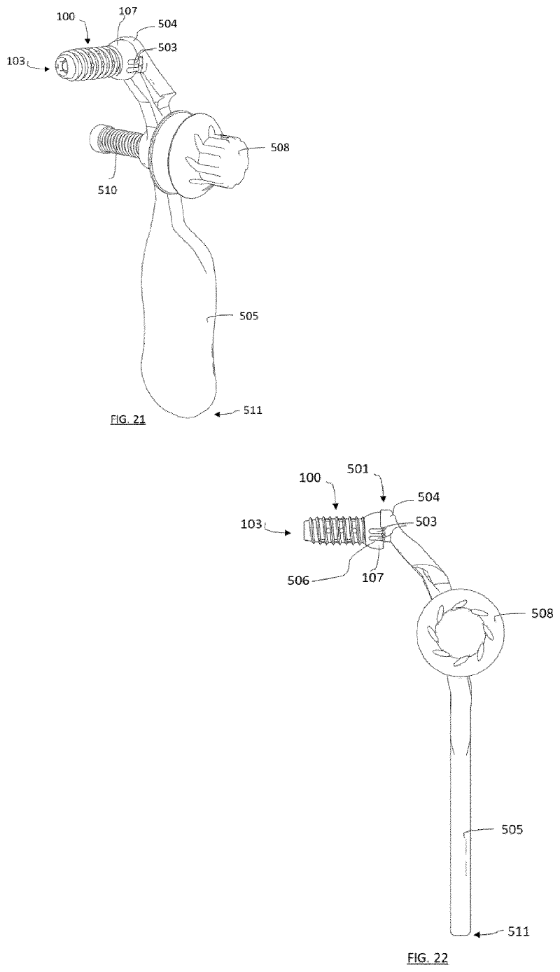

According to a preferred embodiment, the ancillary device 500 comprises a plurality of connecting elements. As a preferred example, the connecting element is a protrusion 503 extending from the distal part in a direction parallel to the axis of the opening 502. The protrusion 503 is preferably positioned around the perimeter of the opening 502 so as to keep the entire section of the opening 502 clear for the passage of the tool for installing the internal part 200. According to one possibility, the ancillary device 500 comprises four protrusions 503 as shown in FIGS. 14 to 22.

According to this embodiment, the additional connecting elements are recesses 506 with a shape complementary to the protrusions 503. The recesses 506 are advantageously formed in the proximal portion 107 and more particularly at the collar. The recesses are formed, for example, at least partially within the thickness of the proximal portion 107, as illustrated in FIGS. 20 to 22, where the recesses 506 are created on the outer surface of the proximal portion 107, with each recess 506 having a shape that is at least partially complementary to a protrusion 503.

According to a preferred possibility, the ancillary device comprises a stop 504 formed on the distal part 501. The stop 504 has a shape that complements a portion of the circumference of the proximal portion 107 of the cage 100, specifically at the circumference of the collar. The stop 504 is preferably formed around the circumference of the opening 502. The stop 504 is, for instance, positioned at the distal end of the distal part 501. The stop 504 advantageously faces the protrusions 503.

According to one possibility, the fixation method of a system according to the invention for ligamentoplasty, as described above, involves the use of the ancillary device 500. The method advantageously comprises drilling a hole in the bone. The process involves inserting the cage 100 into a hole that has been previously drilled in the bone. The setup is advantageously carried out by the drive system 300 as described above and illustrated in FIGS. 10 to 13. Once the cage 100 has been implanted, the graft 400 is passed through the cage 100, specifically through the borehole 101. The graft 400 is advantageously positioned at the proximal end 102 within the notch 112 of the proximal portion 107. The internal part 200 is then installed in the cage 100. The distal end 205 of the internal part 200 is inserted into the borehole 101, preferably while keeping the graft in the notch 112. The ancillary device 500 is then installed; the connecting element of the ancillary device 500 secures into the additional connecting element of the cage 100, specifically the stop 504, and the protrusions 503 are positioned on the proximal portion 107 of the cage 100. The graft 400 is inserted into the slot 507 of the clamping assembly of the ancillary device 500. The clamping knob 508 is actuated to tension the graft 400. Then, preferably using a tool, not shown, designed to engage with the indent 207 of the internal part 200, and passing through the opening 502 of the ancillary device, the internal part 200 is clamped. The proximal part 203, and more specifically the thread 204, advantageously of the subpart 203b, engages with the thread 104 of the proximal portion 107. The internal part 200 is screwed into the cage 100. The internal part 200, and more specifically the distal part 201, is applied to the graft 400 which is itself applied to the inner surface 115 of the distal portion 105 of the cage 100, thus ensuring the fixation of the graft 400. The installation tool and the ancillary device 500 can be removed.

The invention is not limited to the embodiments previously described and extends to all the embodiments covered by the invention.

### LIST OF REFERENCES

1. System
100. Cage
101. Borehole
102. Proximal end
103. Distal end
104. Thread
105. Distal portion
106. Medial portion
107. Proximal portion
109. Angular sector with thread
110. Angular sector without thread
111. Thread
112. Notch
113. Lights
114. Longitudinal axis of the cage
115. Inner surface of the cage
200. Internal part
201. Distal part
202. Central part
203. Proximal part
203a. First subpart
203b. Second subpart
204. Thread
205. Distal end
206. Proximal end
207. Indent
208. Longitudinal axis of the internal part
300. Drive system
301. Distal end
302. Cylindrical area
303. Oblong area
304. Proximal end
305. Additional area of the borehole
306. Intermediate area
307. Stop surface
400. Graft
500. Ancillary device
501. Distal part
502. Opening
503. Protrusions
504. Stop
505. Handle
506. Recesses
507. Slot
508. Clamping knob
509. Clamping axis
510. Clamping spring
511. Proximal part

1. A fixation system for ligamentoplasty comprising:
A cage comprising
i. a longitudinal borehole comprising a proximal end and a distal end,
ii. an outer surface comprising a thread,
iii. a distal portion wherein the borehole is tapered,
iv. a medial portion,
an internal part designed to be inserted into the borehole of the cage comprising
i. a tapered distal part,
ii. a central part,
iii. a proximal part comprising a thread, wherein the cage comprises a proximal portion with a cross-section larger than that of the medial portion thereby forming a collar, and wherein the borehole has a cross-section larger than that of the medial portion, the borehole comprising a thread over a continuous angular sector of less than 360° designed to engage with the internal part.

2. The fixation system according to claim 1, wherein the proximal portion of the cage has an outer surface that has no thread.

3. The fixation system according to claim 1, wherein the proximal portion of the cage comprises an enlargement of the borehole at the proximal end within a complementary angular sector of the angular sector that contains the thread, thereby forming a notch.

4. The fixation system according to claim 1, wherein the angular sector of the borehole that comprises the thread is between 90° and 270°.

5. The fixation system according to claim 1, wherein the borehole is bored in the distal portion and in the medial portion.

6. The fixation system according to claim 1, wherein the distal part of the internal part is smooth.

7. The fixation system according to claim 1, wherein the central part of the internal part is at least partially cylindrical and smooth, and has a smaller cross-section than that of the distal part.

8. The fixation system according to claim 1, wherein the proximal part of the internal part has a cross-section larger than that of the central part.

9. The fixation system according to claim 1, wherein the cage comprises fenestrations designed for the osseointegration of the system.

10. A kit for ligamentoplasty comprising a system according to claim 1 and a drive system for the cage, which comprises a complementary distal end of the proximal portion of the cage having a section with a cylindrical area which corresponds to the angular sector provided with the thread, and an oblong area corresponding to the angular sector having no thread.

11. The kit for ligamentoplasty according to claim 10 comprising an ancillary device comprising at least one connecting element to the proximal portion of the cage and a distal opening designed to guide a tool for securing the internal part.

12. The kit for ligamentoplasty according to claim 11, wherein the ancillary device comprises a plurality of connecting elements arranged around the perimeter of the distal opening, and the cage comprises a plurality of complementary connecting elements designed to engage with the plurality of connecting elements.

13. The kit for ligamentoplasty according to claim 10, wherein the ancillary device comprises a tensioning member for the graft.
